(19) United States
(12) Patent Application Publication
(10) Pub. No.: US 2024/0274238 A1
(43) Pub. Date: Aug. 15, 2024

(54) Deep Learning Model for Predicting a Proteins Ability to Form Pores

(71) Applicant: BASF Agricultural Solutions Seed US LLC, Florham Park, NJ (US)

(72) Inventors: Theju Jacob, Morrisville, NC (US); Theodore Kahn, Morrisville, NC (US)

(73) Assignee: BASF Agricultural Solutions Seed US LLC, Florham Park, NJ (US)

(21) Appl. No.: 18/566,698

(22) PCT Filed: Jun. 9, 2022
(86) PCT No.: PCT/US2022/032815

§ 371 Date: Dec. 4, 2023

Related U.S. Application Data
(60) Provisional application No. 63/209,375, filed on Jun. 10, 2021.

(51) Int. Cl. G16B 35/10; G16B 40/20

(52) U.S. Cl. CPC G16B 35/10; G16B 40/20

(57) ABSTRACT

The following relates generally to identifying pore-forming proteins. In some embodiments, one or more processors: build a training dataset by encoding a first plurality of proteins into numbers; train a deep learning algorithm using the training dataset; encode a second plurality of proteins into numbers; and identify, via the deep learning algorithm, proteins of the encoded second plurality of proteins as either potentially pore-forming or potentially non-pore-forming.

### RELATED APPLICATIONS

This application claims the benefit of U.S. Provisional Application Ser. No. 63/209,375 filed Jun. 10, 2021, the contents of which are herein incorporated by reference in its entirety.

### FIELD OF THE INVENTION

This invention relates to the field of molecular biology and the creation of computational predictive molecular models.

### BACKGROUND OF THE INVENTION

Pore-forming proteins are often used in insecticides. In particular, an insect that ingests a pore-forming protein will develop pores in its gut cell membranes, which will cause death of the insect.

In this regard, various techniques have been developed to identify new pore-forming proteins. However, current techniques have major drawbacks because they: 1) identify dependencies only between amino acids that are within short distances along the protein, and/or 2) identify only pore-forming proteins that are fairly similar to already known pore-forming proteins.

The systems and methods described herein solve these problems and others.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one aspect, a computer-implemented method may be provided. The method may include: building, via one or more processors, a training dataset by encoding a first plurality of proteins into numbers; training, via the one or more processors, a deep learning algorithm using the training dataset; encoding, via the one or more processors, a second plurality of proteins into numbers; and identifying, via the one or more processors and the trained deep learning algorithm, proteins of the encoded second plurality of proteins as either potentially pore-forming or potentially non-pore-forming.

In another aspect, a computer system may be provided. The computer system may include one or more processors configured to: build a training dataset by encoding a first plurality of proteins into numbers; train a deep learning algorithm using the training dataset; encode a second plurality of proteins into numbers; and identify, via the deep learning algorithm, proteins of the encoded second plurality of proteins as either potentially pore-forming or potentially non-pore-forming.

In yet another aspect, another computer system may be provided. The computer system may include: one or more processors; and one or more memories coupled to the one or more processors. The one or more memories may include computer executable instructions stored therein that, when executed by the one or more processors, cause the one or more processors to: build a training dataset by encoding a first plurality of proteins into numbers; train a deep learning algorithm using the training dataset; encode a second plurality of proteins into numbers; and identify, via the deep learning algorithm, proteins of the encoded second plurality of proteins as either potentially pore-forming or potentially non-pore-forming.

### BRIEF DESCRIPTION OF THE DRAWINGS

FIG. 1 shows an example system for determining a pore-forming protein, and/or building an insecticide.

FIG. 2 illustrates an example outline of a deep learning model in accordance with the systems and methods described herein.

FIG. 3 illustrates example accuracy and loss curves for the different encoding methods.

FIG. 4 illustrates example rate of change (ROC) curves for combined one-hot encoding and amino acid feature encoding methods.

FIG. 5 illustrates example receiver operating characteristic curves of the combined encoding method.

FIG. 6 illustrates a flowchart of an example method.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

Embodiments described herein relate to techniques for identifying potentially pore-forming proteins, and for building insecticides.

### INTRODUCTION

Pore-forming proteins form conduits in cell plasma membranes, allowing intracellular and extracellular solutes to leak across cell boundaries. Although the amino acid sequences and three-dimensional structures of pore-forming proteins are extremely diverse, they share a common mode of action in which water-soluble monomers come together to form oligomeric pre-pore structures that insert into membranes to form pores [Sequence Diversity in the Pore-Forming Motifs of the Membrane-Damaging Protein Toxins. Mondal A K, Verma P, Lata K, Singh M, Chatterjee S, Chattopadhyay K. s.l.: J Membr Biol., 2020]. Many pore formers originating from pathogenic bacteria are well documented to be toxic against agricultural pests [Structure, diversity, and evolution of protein toxins from spore-forming entomopathogenic bacteria. de Maagd R. A., Bravo A., Berry C., Crickmore N., Schnepf H. E. 2003, Annual Review of Genetics] [Bacillus thuringiensis Toxins: An Overview of Their Biocidal Activity. Palma, L., Muñoz, D., Berry, C., Murillo, J., and Caballero, P. 2014, Toxins, pp. 3296-3325]. They operate by forming pores in the gut cell membranes of the pests once ingested, causing the death of the pests.

In this regard, orally active pore formers are the key ingredients in several pesticidal products for agricultural use, including transgenic crops. A wide variety of pore-forming protein families are needed for this application for two reasons. First, any given pore former is typically only active against a small number of pest species [Specificity determinants for Cry insecticidal proteins: Insights from their mode of action. N., Jurat-Fuentes J. L. and Crickmore. s.l.: J Invertebr Pathol, 2017]. As a result, proteins from more than one family may be needed to protect a crop from its common pests. Second, the wide-spread use of a particular protein can lead to the development of pests that are resistant to that protein [An Overview of Mechanisms of Cry Toxin Resistance in Lepidopteran Insects. Peterson B., Bezuidenhout C. C, Van den Berg J. 2, s.l.: J Econ Entomol, 2017, Vol. 110] [Insect resistance to Bt crops: lessons from the first billion acres. Tabashnik, B., Brévault, T. and Carrière, Y. s.l.: Nat Biotechnol, 2013, Vol. 31] [Application of pyramided traits against Lepidoptera in insect resistance management for Bt crops. Storer N. P., Thompson G. D., Head G. P. 3, s.l.: GM Crops Food, 2012, Vol. 3]. There is hence an urgent need to identify novel pore formers that can then be developed into new products that will control a broader range of pests, and will delay the development of resistance in pests. A pore former with a new mode of action would overcome resistance; and combining multiple modes of action in one product can delay the development of resistance. Novel pore formers are difficult to find by traditional methods, which involve feeding bacterial cultures to pests, or searching for homologs of known pore formers [Discovery of novel bacterial toxins by genomics and computational biology. Doxey, A. C., Mansfield, M. J., Montecucco, C. 2018, Toxicon]. Modern genome sequencing methods have generated a vast untapped resource of genes whose function is unknown [Hidden in plain sight: what remains to be discovered in the eukaryotic proteome? Wood V., Lock A., Harris M. A., Rutherford K., Bähler J., and Oliver S. G. s.l.: Open Biol., 2019] [Automatic Assignment of Prokaryotic Genes to Functional Categories Using Literature Profiling. Torrieri, R., Silva de Oliveira, F., Oliveira, G., and Coimbra, R. s.l.: Plos One, 2012] [Unknown’ proteins and ‘orphan’ enzymes: the missing half of the engineering parts list—and how to find it. Hanson, A., Pribat, A., Waller, J., and Crécy-Lagard, V. 1, s.l.: The Biochemical journal, 2009, Vol. 425]. Since testing more than a tiny fraction of them for pore-forming activity experimentally is not feasible, computational methods are needed to prioritize which of these proteins should be tested.

The current computational methodology for detecting novel pore-forming proteins relies on sequence homology-based approaches. Sequences of entire proteins and of protein domains from known pore-forming proteins are compared with those proteins whose functionality is unknown, and those that are similar to known toxins are shortlisted for further testing. Basic local alignment search tool (BLAST) [Basic local alignment search tool. Altschul S. F., Gish W., Miller W., Myers E. W., Lipman D. J. 1990, J Mol Biol., pp. 403-410] and Hidden Markov Models (HMM) [Profile hidden Markov models. Eddy, S. R. 9, 1998, Bioinformatics, Vol. 14, pp. 755-763] are the most widely employed tools for sequence homology comparisons. However, these methods 1) identify only dependencies between amino acids that are within short distances along the protein sequence, and 2) identify only sequences that are fairly similar to already existing pore formers. Truly novel pore formers may be sufficiently different from known pore formers that these methods would not identify them.

The systems and methods described herein enable to move beyond sequence homology in detecting potential new pore-forming toxins in the absence of 3-dimensional structural data for either the known or the potentially novel toxins. Broadly speaking, deep learning models have been used for a variety of tasks related to proteins [DeepGO: predicting protein functions from sequence and interactions using a deep ontology-aware classifier. Kulmanov M, Khan M A, Hoehndorf R, Wren J. 2018, Bioinformatics, pp. 660-668.] [Beyond Homology Transfer: Deep Learning for Automated Annotation of Proteins. Nauman, M., Ur Rehman, H., Politano, G. et al. 2019, J Grid Computing, pp. 225-237] [DeepSF: deep convolutional neural network for mapping protein sequences to folds. Hou J, Adhikari B, Cheng J. 2018, Bioinformatics, pp. 1295-1303] [DEEPred: Automated Protein Function Prediction with Multi-task Feed-forward Deep Neural Networks. Sureyya Rifaioglu, A., Doğan, T., Jesus Martin, M. et al. 2019, Nature Scientific Reports] [Predicting the sequence specificities of DNA-and RNA-binding proteins by deep learning. Alipanahi, B., Delong, A., Weirauch, M. et al. 2015, Nature Biotechnology, pp. 831-838].

Some embodiments leverage deep learning to capture not just dependencies between neighboring amino acids as is done in traditional sequence matching methods such as HMMs, but also dependencies between amino acids that are farther apart along the protein sequence. By encoding amino acids in terms of their physical and chemical properties, some embodiments capture the basic characteristics of a protein that form pores, allowing us to identify novel pore formers based on similarities that currently are not recognized.

Pore-forming proteins may be broadly classified into alpha and beta categories based on the secondary structures of their membrane spanning elements [Pore-forming protein toxins: from structure to function. Parker, M. W., and Feil, S. C. 2005, Progress in Biophysics and Molecular Biology, pp. 91-142] [Pore-forming toxins: ancient, but never really out of fashion. Peraro, M. D. and van der Goot, F. G. 2016, Nature Reviews]. For instance, an alpha pore-forming protein may include an alpha helix secondary structure, and a beta pore-forming protein may include a beta barrel secondary structure. Examples of pesticidal alpha pore formers include multiple Cry protein family members and Vip3 protein family members, while examples of pesticidal beta pore formers include Mtx and Toxin 10 protein family members [A structure-based nomenclature for Bacillus thuringiensis and other bacteria derived pesticidal proteins. Crickmore, N., Berry, C., Panneerselvam, S., Mishra, R., Connor, T., and Bonning, B. s.l.: Journal of Invetebrate Pathology, 2020] [Pore-forming protein toxins: from structure to function. Parker, M. W., and Feil, S. C. 2005, Progress in Biophysics and Molecular Biology, pp. 91-142].

Some implementations distinguish pore-forming proteins from non-pore-forming proteins, regardless of whether they are alpha or beta pore-forming proteins. Some embodiments use publicly available data of sequences of alpha and beta pore-forming proteins [e.g., Uniprot. Uniprot. [Online] https://www.uniprot.org/] as part of the training set for a deep learning model. Some implementations use a series of encoding methods for the proteins in the training set, and evaluate their accuracy in distinguishing pore forming from non-pore forming proteins. Some embodiments also evaluate the precision and recall characteristics of these encoding methods. In addition, comparisons may be made to BLAST and HMM models when attempting to detect pore formers that were not part of the training set.

### Experimental Examples

### Infrastructure

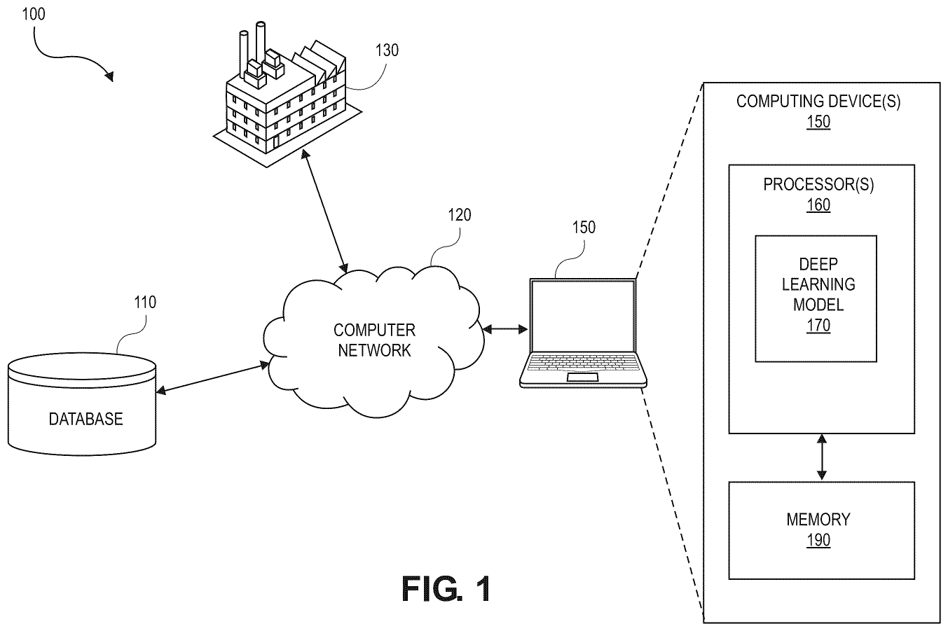

FIG. 1 shows an example system 100. With reference thereto, computing device 150 (e.g., a computer, tablet, server farm, etc.) may be connected to computer network 120 through base station 110. Computer network 120 may comprise a packet based network operable to transmit computer data packets among the various devices and servers described herein. For example, computer network 120 may consist of any one or more of Ethernet based network, a private network, a local area network (LAN), and/or a wide area network (WAN), such as the Internet.

With further reference to FIG. 1, the computing device 150 is connected to the computer network 120. As is understood in the art, the computing device(s) includes processor(s) and memory. In the example of FIG. 1, the computing device 150 includes processor(s) 160 (which includes deep learning model 170, as described below) and memory 190. As is understood in the art, the processor 160 may be a single processor or as a group of processors. Furthermore, the deep learning model 170 may be implemented on a single processor or group of processors.

The example of FIG. 1 also illustrates database 110. In some embodiments, the database 110 includes a database of pore-forming protein data. Although the example of FIG. 1 illustrates the database 110 separately from the computing device 150, in some implementations, the database 110 is part of the computing device 150 (e.g., part of the memory 190, or separate from the memory 190).

Further illustrated in the example of FIG. 1 is factory 130 (e.g., an insecticide factory). In some embodiments, the computing device 150 identifies a pore-forming protein, and the factory 130 manufactures the pore forming protein or an insecticide including the pore-forming protein. In some embodiments, the computing device 150 determines the entire insecticide formula including the pore-forming protein. In other embodiments, the computing device 150 determines only the pore-forming protein, and the complete insecticide formula is determined by the factory 130 (e.g., by computers, servers, etc. of the factory 130).

### Model

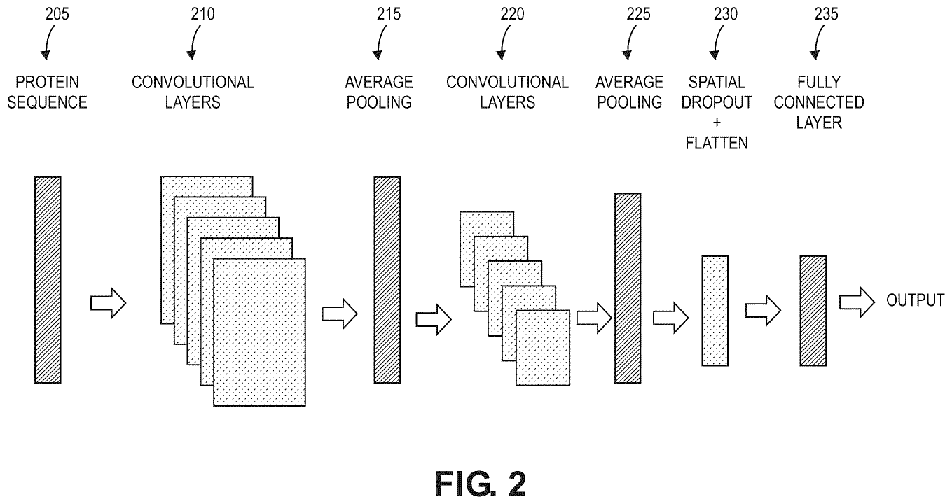

One example of the outline of the deep learning model is as shown in FIG. 2. The encoded protein sequence 205 passes through multiple convolutional layers 210, 220, and pooling layers 215, 225. It is then followed by a dropout layer 230, after which it is passed through a fully connected layer 235 to the output. In some embodiments, the hyperparameters of the network are selected by Bayesian optimization.

In some embodiments, the encoded protein sequence 210 is fed to first convolutional layer 210 with 25 filters of dimensions 1×100; and second convolutional layer 220 with a set of convolutional layer filters having dimensions 1×50. In some embodiments, a Rectified Linear Unit (ReLU) was used as the activation function. In some implementations, mean squared error was the metric used as the loss function. In some implementations, the pooling layers had a pool size of 5, and the dropout layer had a factor of 0.25.

### Data

Any data source (e.g., database 110) may be used for alpha and beta pore-forming proteins. Under alpha pore formers, some embodiments include pesticidal crystal proteins, actinoporins, hemolysins, colicins, and perfringolysins. Under beta pore formers, some implementations include leucocidins, alpha-hemolysins, perifringolysins, aerolysins, haemolysins, and cytolysins. Some embodiments begin by initially eliminating all amino acid sequences that are shorter than a first predetermined length (e.g., 50) of amino acids and/or longer than a second predetermined length (e.g., 2000) of amino acids. Some embodiments include both fragments and full proteins in the data set. Some implementations obtain approximately 3000 proteins belonging to both alpha and beta pore-forming families. To avoid overfitting the model 170, some embodiments, before training, cluster the amino acid sequences at 70% identity. Some embodiments use zero padding to ensure all sequences were of the same length before training. This step also enables to avoid multiple sequence alignments that would have rendered the model 170 impractical when eventually testing with millions of proteins (e.g., to generate position specific scoring matrices (PSSMs) for 3000 proteins, it will take over a week).

It is advantageous to cover as much diversity as possible in terms of possible protein structures the model 170 might encounter. Some embodiments use a culled protein data bank (PDB) dataset from the PISCES server [PISCES: a protein sequence culling server. Wang, G., and Dunbrack, Jr. R. L. 2003, Bioinformatics, pp. 1589-1591]. In some implementations, the dataset sequences had less than 20 percent sequence identity, with better than 1.8 Å resolution. In some embodiments, the lengths were once again restricted to fall within the 50-2000 amino acid range. Some implementations eliminated sequences that were similar to the ones in the positive training set, based on BLASTP results with an E-value of 0.01. The final list had approximately 5000 sequences.

### Comparison of Various Encoding Schemes

Protein sequences consist of amino acids, typically denoted by letters. For a computational algorithm to make sense of them, they need to be represented as numbers. A representation of letters along the protein sequence by predetermined numbers will work—for example, every amino acid can be represented by a unique number. Or, they can be one-hot encoded, where every position along a protein sequence is represented by an indicator array, with a one denoting the amino acid in that position, and the rest all zeros. In the literature, a method that has been used is the representation of a combination of, say, amino acids in sets of three (trigrams), by a unique number [DeepGO: predicting protein functions from sequence and interactions using a deep ontology-aware classifier. Kulmanov M, Khan M A, Hoehndorf R, Wren J. 2018, Bioinformatics, pp. 660-668]. Position specific scoring matrices (PSSM) is another used method to obtain numerical representations for protein sequences [Deep Supervised and Convolutional Generative Stochastic Network for Protein Secondary Structure Prediction. Zhou, J., and Troyanskaya, O. s.l.: Proceedings of the 31st International Conference on International Conference on Machine Learning, 2014].

Some embodiments represent protein sequences by an encoding method that enables to eventually test the model 170 with millions of test proteins. These embodiments thus rule out methods that require comparisons with existing protein databases, such as PSSMs. Some embodiments also rule out utilizing domain information from known pore formers, to avoid biasing the model 170 towards already known proteins. One-hot encoding would allow to rapidly convert the amino acid sequences to numbers, but it treats all amino acids the same, thus requiring a larger dimensional space.

In this regard, certain advantages may be achieved by finding a technique of representing amino acids that captures their properties in as low dimensional a space as possible. One known technique [Solving the protein sequence metric problem. Atchley, W. R., Zhao, J., Fernandes, A. D., and Druke, T. 2005, Proceedings of the National Academy of Sciences, pp. 6395-6400] selected 54 amino acid attributes were analyzed and reduced to 5 amino acid features. The 5 numbers that corresponded to each amino acid captured are:
Accessibility, polarity, and hydrophobicity
Propensity for secondary structure
Molecular size
Codon composition
Electrostatic charge

Similar numbers along any of these 5 amino acid features indicated similarity in the corresponding property space. Table 1 below shows one example implementation of encoding using this amino acid feature technique (e.g., the 5 amino acid features are illustrated as 5 factors in Table 1).

TABLE 1
Example implementation of the amino acid feature technique for
encoding amino acids (features listed as factors on the table).

| Amino | Factor | Factor | Factor | Factor | Factor |
|---|---|---|---|---|---|
| acid | I | II | III | IV | V |
| A | −0.591 | −1.302 | −0.733 | 1.570 | −0.146 |
| C | −1.343 | 0.465 | −0.862 | −1.020 | −0.255 |
| D | 1.050 | 0.302 | −3.656 | −0.259 | −3.242 |
| E | 1.357 | −1.453 | 1.477 | 0.113 | −0.837 |
| F | −1.006 | −0.590 | 1.891 | −0.397 | 0.412 |
| G | −0.384 | 1.652 | 1.330 | 1.045 | 2.064 |
| H | 0.336 | −0.417 | −1.673 | −1.474 | −0.078 |
| I | −1.239 | −0.547 | 2.131 | 0.393 | 0.816 |
| K | 1.831 | −0.561 | 0.533 | −0.277 | 1.648 |
| L | −1.019 | −0.987 | −1.505 | 1.266 | −0.912 |
| M | −0.663 | −1.524 | 2.219 | −1.005 | 1.212 |
| N | 0.945 | 0.828 | 1.299 | −0.169 | 0.933 |
| P | 0.189 | 2.081 | −1.628 | 0.421 | −1.392 |
| Q | 0.931 | −0.179 | −3.005 | −0.503 | −1.853 |
| R | 1.538 | −0.055 | 1.502 | 0.440 | 2.897 |
| S | −0.228 | 1.399 | −4.760 | 0.670 | −2.647 |
| T | −0.032 | 0.326 | 2.213 | 0.908 | 1.313 |
| V | −1.337 | −0.279 | −0.544 | 1.242 | −1.262 |
| W | −0.595 | 0.009 | 0.672 | −2.128 | −0.184 |
| Y | 0.260 | 0.830 | 3.097 | −0.838 | 1.512 |

In addition to capturing amino acid properties, this representation is attractive as the feature space is comparatively low dimensional. For example, in some embodiments, one-hot encoding represents an amino acid using a 28-dimensional array (all of the amino acids plus characters used for zero padding), while the amino acid feature technique encodes the same amino acid using a 5-dimensional array. A smaller feature space makes the training times and memory requirements of the model much more manageable, but it is advantageous to strike a balance with accuracy and loss metrics as well. Thus, some embodiments use one-hot encoding (e.g., 28 dimensional feature space), amino acid feature encoding (e.g., 5-dimensional feature space), as well as combined one-hot encoding and amino acid feature encoding (e.g., 33 dimensional feature space) methods.

### Results

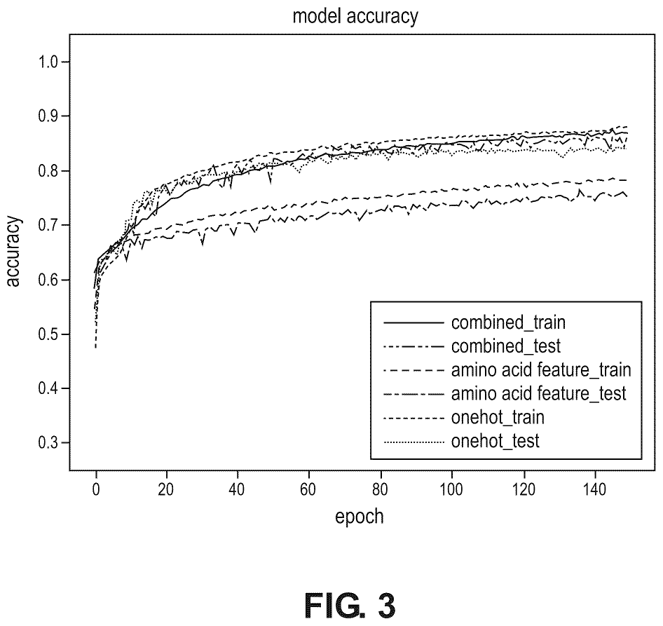

Example accuracy and loss curves for the different encoding methods are shown in FIG. 3. As can be observed, the accuracy and loss curves converged during training of the model. Accuracy values reaching approximately 90% and loss values reaching approximately 5% were observed by end of training. One-hot and the combined encoding methods did better than amino acid feature encoding in terms of both accuracy as well as loss curves. The combined encoding method was comparable to one-hot encoding initially, but towards the end of the training, started to give better performance than one-hot encoding. The data set was split 80:20 for training and validation purposes.

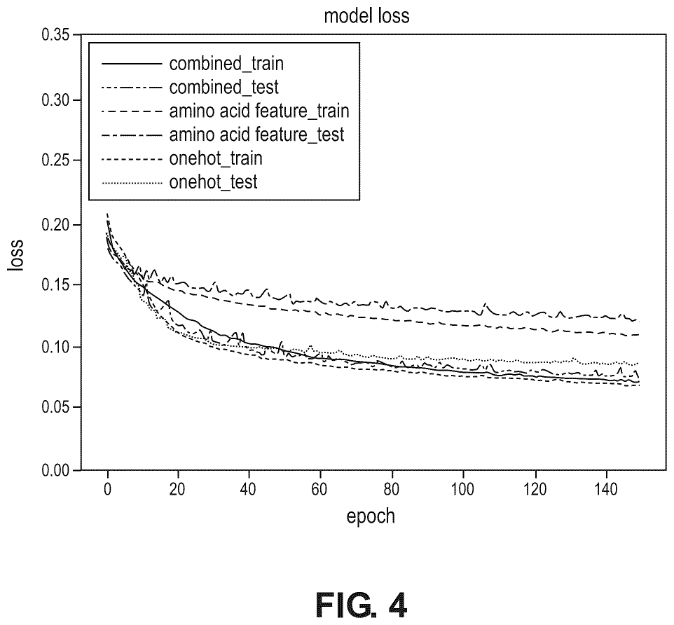

Example rate of change (ROC) curves for combined one-hot encoding and amino acid feature encoding methods are shown in FIG. 4. As can be seen from the curves and the area under the curve (AOC) values, the model gives near ideal performance on the dataset it was trained with.

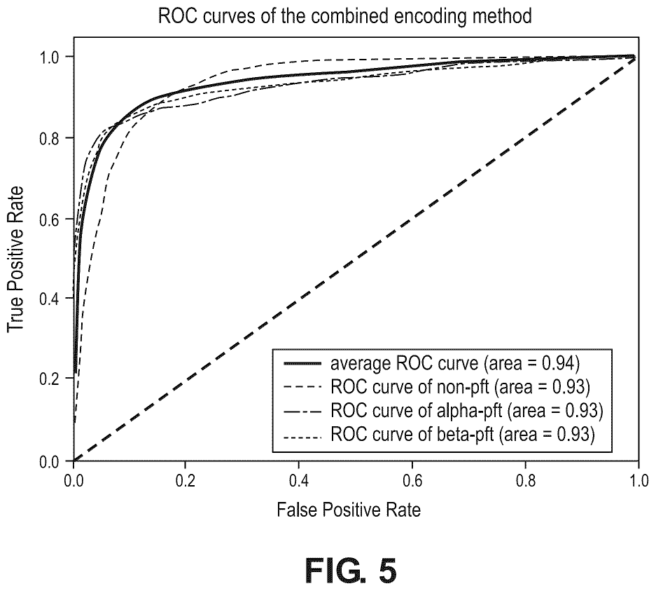

FIG. 5 illustrates example receiver operating characteristic curves of the combined encoding method. In this regard, FIG. 5 illustrates curves for the negative, alpha, and beta pore formers, as well as the average ROC curve.

One goal was to evaluate if the model 170 could pick up novel pore formers it had not seen previously during training, better than standard methods like BLAST and HMM. Towards that end, testing was performed on 3 known pore former families that had not been included during training of the model 170: Vip3, MACPF, and Toxin 10. A comparison of the performance of the model against BLAST and HMM is summarized in Table 2.

Table 2: Table comparing BLAST, HMM, and the disclosed model (e.g., model 170) with the three protein families of interest. The column corresponding to each method shows how many proteins belonging to each category were picked by the corresponding method. The table shows that the disclosed model managed to detect pore formers that were missed by traditional sequence homology approaches.

TABLE 2
Table comparison of BLAST, HMM, and the disclosed models

| | | | Amino acid | | Amino acid |
|---|---|---|---|---|---|
| Protein | BLAST | HMM | feature | One-hot | feature + One-hot |
| Vip3 | 0 | 0 | 95 | 99 | 108 |
| (108) | | | | | |
| MACPF | 1 | 0 | 3 | 3 | 4 |
| (5) | | | | | |
| Toxin 10 | 0 | 0 | 10 | 17 | 21 |
| (30) | | | | | |

For this test data of the sequences of the Vip3, MACPF, and Toxin 10 proteins was taken from the Bacterial Pesticidal Protein Resource Center [BPPRC. [Online] https://www.bpprc.org/.]. The used list of test proteins had 108 Vip3s, 5 MACPFs, and 30 Toxin 10 family proteins. For the tests that were run with the three protein families, no homologs of the three families were present in the training set—that is, no Vip3s or Perforins or Toxin 10s. To evaluate BLAST, a BLAST database was made out of the training set, and compared with the test proteins. The E-value used was 0.01. The single hit for MACPF was due to the presence of thiol-activated cytolysins in the training set. To evaluate HMMs, HMMs were downloaded for each protein category in the training set from the PFAM database [Pfam database. [Online] http://pfam.xfam.org/], and evaluated to determine if any of them could pick up proteins from the test list. The HMMs that were downloaded included aerolysins, leukocidins, anemone_cytotox, colicin, endotoxin_c, endotoxin_h, hemolysin_n, and hlye (Hemolysin E). None of the HMMs considered were able to pick up any of the proteins from the test categories—that is, HMMs are not geared towards picking up novel proteins. For the disclosed deep learning model 170, after training, the model was tested with the list of these proteins, and checked to see how many of these were picked up by the model as pore formers. As the table summarizes, the model 170 managed to detect pore formers it was not trained on, even when traditional sequence homology-based approaches failed. Once again, the combined encoding method outperformed one-hot encoding and amino acid feature 5-factor encoding methods.

### Example Embodiment

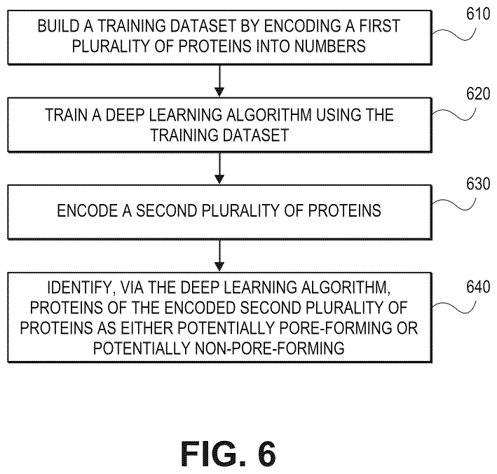

FIG. 6 illustrates a flowchart of an example method. With reference thereto, at block 610, a training dataset it built by encoding a first plurality of proteins into numbers. The encoding may be done by any of the techniques described herein or by any suitable technique.

At block 620, a deep learning algorithm or model 170 is trained using the training dataset. At block 630, a second plurality of proteins is encoded. As with the encoding of the first plurality of proteins, the encoding for the second plurality of proteins may be done by any of the techniques described herein or by any suitable technique. At block 640, via the deep learning algorithm or model 170, proteins of the encoded second plurality of proteins are identified as either potentially pore-forming or potentially non-pore-forming.

It should be understood that the blocks of FIG. 6 do not necessarily need to be performed in the order that they are presented (e.g., the blocks may be performed in any order). Further, additional blocks may be performed in addition to those presented in the example of FIG. 6. Still further, not all of the blocks of FIG. 6 must be performed (e.g., the blocks may be optional in some embodiments).

### Aspects

Aspect 1. A computer-implemented method, comprising:
building, via one or more processors, a training dataset by encoding a first plurality of proteins into numbers;
training, via the one or more processors, a deep learning algorithm using the training dataset;
encoding, via the one or more processors, a second plurality of proteins into numbers; and
identifying, via the one or more processors and the trained deep learning algorithm, proteins of the encoded second plurality of proteins as either potentially pore-forming or potentially non-pore-forming.

Aspect 2. The computer-implemented method of aspect 1, wherein the first plurality of proteins comprises a protein comprising a sequence of amino acids, and wherein the encoding the first plurality of proteins into numbers comprises:
representing each amino acid in the sequence of amino acids as an indicator array, wherein the indicator array indicates a type of amino acid by making a single element of the indicator array either: (i) equal to one, and the rest of the elements equal to zero; or (ii) equal to zero, and the rest of the elements equal to one.

Aspect 3. The computer-implemented method of any of aspects 1-2, wherein the first plurality of proteins comprises a protein comprising a sequence of amino acids, and wherein the encoding the first plurality of proteins into numbers comprises:
representing each amino acid in the sequence of amino acids as an array, wherein elements of the array correspond to amino acid features.

Aspect 4. The computer-implemented method of any of aspects 1-3, wherein the first plurality of proteins comprises a protein comprising a sequence of amino acids, and wherein the encoding the first plurality of proteins into numbers comprises:
representing each amino acid in the sequence of amino acids as an array, wherein elements of the array correspond to amino acid features, and wherein the amino acid attributes comprise:
(i) accessibility, polarity, and hydrophobicity;
(ii) propensity for secondary structure;
(iii) molecular size;
(iv) codon composition; or
(v) electrostatic charge.

Aspect 5. The computer-implemented method of any of aspects 1-4, wherein the first plurality of proteins comprises a protein comprising a sequence of amino acids, and wherein the encoding the first plurality of proteins into numbers comprises:
representing each amino acid in the sequence of amino acids as an array, wherein elements of the array correspond to amino acid features, and wherein the amino acid attributes comprise:
(i) accessibility, polarity, and hydrophobicity;
(ii) propensity for secondary structure;
(iii) molecular size;
(iv) codon composition; and
(v) electrostatic charge.

Aspect 6. The computer-implemented method of any of aspects 1-5, wherein the first plurality of proteins comprises a protein comprising a sequence of amino acids, and wherein the encoding the first plurality of proteins into numbers comprises:
representing each amino acid in the sequence of amino acids as a combined array, wherein the combined array is formed by combining:
   a first array which indicates a type of amino acid by making a single element of the first array either: (i) equal to one, and the rest of the elements equal to zero; or (ii) equal to zero, and the rest of the elements equal to one; and
   a second array with elements of the second array corresponding to amino acid features.

Aspect 7. The computer-implemented method of any of aspects 1-6, wherein the deep learning algorithm comprises a convolutional neural network.

Aspect 8. The computer-implemented method of any of aspects 1-7, wherein the deep learning algorithm comprises a convolutional neural network (CNN), and wherein the CNN comprises:
at least one convolutional layer;
at least one average pooling layer; and
a spatial dropout layer.

Aspect 9. The computer-implemented method of any of aspects 1-8, wherein the identifying the proteins of the encoded second plurality of proteins further comprises identifying proteins as: (i) alpha pore-forming proteins; (ii) beta pore forming proteins, or (iii) neither alpha pore-forming proteins nor beta pore-forming proteins, wherein alpha pore-forming proteins have an alpha helix structure, and beta pore forming proteins have a beta barrel structure.

Aspect 10. The computer-implemented method of any of aspects 1-9, further comprising:
determining, via the one or more processors, an insecticide formula based on a protein of the plurality of proteins identified to be potentially pore-forming; and
manufacturing an insecticide based on the determined insecticide formula.

Aspect 11. A computer system comprising one or more processors configured to:
build a training dataset by encoding a first plurality of proteins into numbers;
train a deep learning algorithm using the training dataset;
encode a second plurality of proteins into numbers; and
identify, via the deep learning algorithm, proteins of the encoded second plurality of proteins as either potentially pore-forming or potentially non-pore-forming.

Aspect 12. The computer system of aspect 11, wherein the first plurality of proteins comprises a protein comprising a sequence of amino acids, and wherein the one or more processors are further configured to encode the first plurality of proteins into numbers by:
representing each amino acid in the sequence of amino acids as an indicator array, wherein the indicator array indicates a type of amino acid by making a single element of the indicator array either: (i) equal to one, and the rest of the elements equal to zero; or (ii) equal to zero, and the rest of the elements equal to one.

Aspect 13. The computer system of any of aspects 11-12, wherein the first plurality of proteins comprises a protein comprising a sequence of amino acids, and wherein the one or more processors are further configured to encode the first plurality of proteins into numbers by:
representing each amino acid in the sequence of amino acids as an array, wherein elements of the array correspond to amino acid features.

Aspect 14. The computer system of any of aspects 11-13, wherein the deep learning algorithm comprises a convolutional neural network (CNN), and wherein the CNN comprises:
at least one convolutional layer;
at least one average pooling layer; and
a spatial dropout layer.

Aspect 15. The computer system of any of aspects 11-14, wherein the one or more processors are further configured to:
determine an insecticide formula based on a protein of the plurality of proteins identified to be potentially pore-forming; and
wherein the computer system further comprises manufacturing equipment configured to manufacture an insecticide based on the insecticide formula.

Aspect 16. A computer system comprising:
one or more processors; and
one or more memories coupled to the one or more processors;
the one or more memories including computer executable instructions stored therein that, when executed by the one or more processors, cause the one or more processors to:
build a training dataset by encoding a first plurality of proteins into numbers;
train a deep learning algorithm using the training dataset;
encode a second plurality of proteins into numbers; and
identify, via the deep learning algorithm, proteins of the encoded second plurality of proteins as either potentially pore-forming or potentially non-pore-forming.

Aspect 17. The computer system of aspect 16, wherein the first plurality of proteins comprises a protein comprising a sequence of amino acids, and wherein the computer executable instructions, when executed by the one or more processors, further cause the one or more processors to encode the first plurality of proteins into numbers by:
representing each amino acid in the sequence of amino acids as an indicator array, wherein the indicator array indicates a type of amino acid by making a single element of the indicator array either: (i) equal to one, and the rest of the elements equal to zero; or (ii) equal to zero, and the rest of the elements equal to one.

Aspect 18. The computer system of any of aspects 16-17, wherein the first plurality of proteins comprises a protein comprising a sequence of amino acids, and wherein the computer executable instructions, when executed by the one or more processors, further cause the one or more processors to encode the first plurality of proteins into numbers by:
representing each amino acid in the sequence of amino acids as an array, wherein elements of the array correspond to amino acid features.

Aspect 19. The computer system of any of aspects 16-18, wherein the deep learning algorithm comprises a convolutional neural network (CNN), and wherein the CNN comprises:
at least one convolutional layer;
at least one average pooling layer; and
a spatial dropout layer.

Aspect 20. The computer system of any of aspects 16-19, wherein the computer executable instructions, when executed by the one or more processors, further cause the one or more processors to:
determine an insecticide formula based on a protein of the plurality of proteins identified to be potentially pore-forming; and
wherein the computer system further comprises manufacturing equipment configured to manufacture an insecticide based on the insecticide formula.

### Other Matters

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of geographic locations.

That which is claimed:

1. A computer-implemented method, comprising the steps of:
a) building, via one or more processors, a training dataset by encoding a first plurality of proteins into numbers;
b) training, via the one or more processors, a deep learning algorithm using the training dataset;
c) encoding, via the one or more processors, a second plurality of proteins into numbers; and
d) identifying, via the one or more processors and the trained deep learning algorithm, proteins of the encoded second plurality of proteins as either potentially pore-forming or potentially non-pore-forming.

2. The computer-implemented method of claim 1, wherein the first plurality of proteins comprises a protein comprising a sequence of amino acids, and wherein the encoding the first plurality of proteins into numbers comprises representing each amino acid in the sequence of amino acids as an indicator array, wherein the indicator array indicates a type of amino acid by making a single element of the indicator array either: (i) equal to one, and the rest of the elements equal to zero; or (ii) equal to zero, and the rest of the elements equal to one.

3. The computer-implemented method of claim 1, wherein the first plurality of proteins comprises a protein comprising a sequence of amino acids, and wherein the encoding the first plurality of proteins into numbers comprises representing each amino acid in the sequence of amino acids as an array, wherein elements of the array correspond to amino acid features.

4. The computer-implemented method of claim 1, wherein the first plurality of proteins comprises a protein comprising a sequence of amino acids, and wherein the encoding the first plurality of proteins into numbers comprises representing each amino acid in the sequence of amino acids as an array, wherein elements of the array correspond to amino acid features, and wherein the amino acid attributes comprise:
(i) accessibility, polarity, and hydrophobicity;
(ii) propensity for secondary structure;
(iii) molecular size;
(iv) codon composition; or
(v) electrostatic charge.

5. The computer-implemented method of claim 1, wherein the first plurality of proteins comprises a protein comprising a sequence of amino acids, and wherein the encoding the first plurality of proteins into numbers comprises representing each amino acid in the sequence of amino acids as an array, wherein elements of the array correspond to amino acid features, and wherein the amino acid attributes comprise:
(i) accessibility, polarity, and hydrophobicity;
(ii) propensity for secondary structure;
(iii) molecular size;
(iv) codon composition; and
(v) electrostatic charge.

6. The computer-implemented method of claim 1, wherein the first plurality of proteins comprises a protein comprising a sequence of amino acids, and wherein the encoding the first plurality of proteins into numbers comprises representing each amino acid in the sequence of amino acids as a combined array, wherein the combined array is formed by combining a first array which indicates a type of amino acid by making a single element of the first array either: (i) equal to one, and the rest of the elements equal to zero; or (ii) equal to zero, and the rest of the elements equal to one; and a second array with elements of the second array corresponding to amino acid features.

7. The computer-implemented method of claim 1, wherein the deep learning algorithm comprises a convolutional neural network.

8. The computer-implemented method of claim 1, wherein the deep learning algorithm comprises a convolutional neural network (CNN), and wherein the CNN comprises at least one convolutional layer; at least one average pooling layer; and a spatial dropout layer.

9. The computer-implemented method of claim 1, wherein the identifying the proteins of the encoded second plurality of proteins further comprises identifying proteins as: (i) alpha pore-forming proteins; (ii) beta pore forming proteins, or (iii) neither alpha pore-forming proteins nor beta pore-forming proteins, wherein alpha pore-forming proteins have an alpha helix structure, and beta pore forming proteins have a beta barrel structure.

10. The computer-implemented method of claim 1, further comprising: determining, via the one or more processors, an insecticide formula based on a protein of the plurality of proteins identified to be potentially pore-forming; and manufacturing an insecticide based on the determined insecticide formula.

11. A computer system comprising one or more processors configured to build a training dataset by encoding a first plurality of proteins into numbers; train a deep learning algorithm using the training dataset; encode a second plurality of proteins into numbers; and identify, via the deep learning algorithm, proteins of the encoded second plurality of proteins as either potentially pore-forming or potentially non-pore-forming.

12. The computer system of claim 11, wherein the first plurality of proteins comprises a protein comprising a sequence of amino acids, and wherein the one or more processors are further configured to encode the first plurality of proteins into numbers by representing each amino acid in the sequence of amino acids as an indicator array, wherein the indicator array indicates a type of amino acid by making a single element of the indicator array either: (i) equal to one, and the rest of the elements equal to zero; or (ii) equal to zero, and the rest of the elements equal to one.

13. The computer system of claim 11, wherein the first plurality of proteins comprises a protein comprising a sequence of amino acids, and wherein the one or more processors are further configured to encode the first plurality of proteins into numbers by representing each amino acid in the sequence of amino acids as an array, wherein elements of the array correspond to amino acid features.

14. The computer system of claim 11, wherein the deep learning algorithm comprises a convolutional neural network (CNN), and wherein the CNN comprises: at least one convolutional layer; at least one average pooling layer; and a spatial dropout layer.

15. The computer system of claim 11, wherein the one or more processors are further configured to determine an insecticide formula based on a protein of the plurality of proteins identified to be potentially pore-forming; and wherein the computer system further comprises manufacturing equipment configured to manufacture an insecticide based on the insecticide formula.

16. A computer system comprising one or more processors; and one or more memories coupled to the one or more processors; the one or more memories including computer executable instructions stored therein that, when executed by the one or more processors, cause the one or more processors to: build a training dataset by encoding a first plurality of proteins into numbers; train a deep learning algorithm using the training dataset; encode a second plurality of proteins into numbers; and identify, via the deep learning algorithm, proteins of the encoded second plurality of proteins as either potentially pore-forming or potentially non-pore-forming.

17. The computer system of claim 16, wherein the first plurality of proteins comprises a protein comprising a sequence of amino acids, and wherein the computer executable instructions, when executed by the one or more processors, further cause the one or more processors to encode the first plurality of proteins into numbers by representing each amino acid in the sequence of amino acids as an indicator array, wherein the indicator array indicates a type of amino acid by making a single element of the indicator array either: (i) equal to one, and the rest of the elements equal to zero; or (ii) equal to zero, and the rest of the elements equal to one.

18. The computer system of claim 16, wherein the first plurality of proteins comprises a protein comprising a sequence of amino acids, and wherein the computer executable instructions, when executed by the one or more processors, further cause the one or more processors to encode the first plurality of proteins into numbers by representing each amino acid in the sequence of amino acids as an array, wherein elements of the array correspond to amino acid features.

19. The computer system of claim 16, wherein the deep learning algorithm comprises a convolutional neural network (CNN), and wherein the CNN comprises at least one convolutional layer; at least one average pooling layer; and a spatial dropout layer.

20. The computer system of claim 16, wherein the computer executable instructions, when executed by the one or more processors, further cause the one or more processors to determine an insecticide formula based on a protein of the plurality of proteins identified to be potentially pore-forming; and wherein the computer system further comprises manufacturing equipment configured to manufacture an insecticide based on the insecticide formula.
